(19) United States
(12) Patent Application Publication
(10) Pub. No.: US 2008/0175921 A1
(43) Pub. Date: Jul. 24, 2008

(54) Colloidal formulation of long-acting insulin and its preparation

(76) Inventors: Alain Constancis, Lyon (FR); David Duracher, Lyon (FR); Olivier Soula, Meyzieu (FR); Nathan Bryson, Millery (FR)

Correspondence Address: PATTON BOGGS LLP, 8484 WESTPARK DRIVE, SUITE 900, MCLEAN, VA, 22102, US

(21) Appl. No.: 11/907,039

(22) Filed: Oct. 9, 2007

Related U.S. Application Data
(63) Continuation of application No. 11/654,545, filed on Jan. 18, 2007, now abandoned.
(60) Provisional application No. 60/759,576, filed on Jan. 18, 2006.

(30) Foreign Application Priority Data
Jan. 18, 2006 (FR) 06 50183
Jan. 16, 2007 (EP) PCT/EP2007/000335

(51) Int. Cl. A61K 38/28; A61P 3/10; A61K 9/107

(52) U.S. Cl. 424/501; 514/3

(57) ABSTRACT

The invention relates to injectable long-acting insulin formulations for the treatment of type I and II diabetes in humans and animals.

The main objective of the invention is to provide a long-acting insulin formulation in the form of a colloidal suspension:
which allows easy filling of a syringe through a small diameter needle (for example with the gauge 29 G, 30 G or 31 G)
and/or which can be easily injected through a small diameter needle (for example with the gauge 29 G, 30 G or 31 G),
   without damaging the therapeutic efficacy of the insulin.

To achieve this objective, the subject of the invention is an aqueous and stable colloidal formulation of nanoparticles of at least one poly(Leu-block-Glu), loaded with insulin, in which the pH is such that: 6.0≦pH≦7.0

which comprises at least one magnesium salt in a quantity such that:
the osmolarity Osm (in mOsmol) is such that: 270≦Osm≦600,
the viscosity v (in mPa·s), measured according to a procedure Mv, is such that: v≦15;
the poly(Leu-block-Glu) concentration (in mg/ml) is between 30 and 70, preferably between 38 and 65.

The field of the present invention is that of medicaments based on insulin, in particular injectable insulin formulations, for the treatment of type I and II diabetes in humans and animals.

The present invention relates, more precisely, to injectable insulin formulations made of colloidal suspensions of insulin intended for daily parenteral administration and capable of maintaining, during the entire nychthemeron in the diabetic patient or animal, a plasma insulin concentration close to the basal concentration observed in a nondiabetic subject.

The invention also relates to the methods for preparing said colloidal suspensions of insulin.

Diabetics are subjected to a very inconvenient and imperfect treatment, forcing them to self-inject insulin several times per day. In addition to the rapidly absorbed insulin injections, intended to correct the rise in glycemia after or during a meal, it is also necessary to maintain the serum insulin at the basal level day and night in order to avoid very harmful side effects. The latter correction is particularly delicate because during the night, patients do not have the opportunity to treat themselves and thus to reestablish the desirable level of insulin.

Injectable insulin formulations should be stable, namely that the insulin which they contain should not undergo degradation during storage. This insulin should for example be always fully effective after 2 years of storage at 5° C. Injectable insulin formulations should have a rheology suited to the injection systems commonly used for insulin. An acute need therefore exists for a long-acting insulin formulation which can be injected through very fine needles and which is stable. The problem of developing such a formulation has been known for a long time and has been the subject of numerous studies.

For the purposes of the present disclosure, “insulin” denotes both human insulin or an animal insulin or alternatively an insulin analog.

For the purposes of the present disclosure, a “long-acting insulin formulation” is a formulation which makes it possible, on the one hand, to avoid, after administration, any hypoglycemia peak that is harmful for the patient and, on the other hand, to maintain a hypoglycemic action over at least 24 hours.

### PRIOR ART

Mention is made hereinafter to a number of previous technical proposals for long-acting insulin formulations.

Thus, long-acting human insulin, NPH, is known for example. It consists of partially microcrystallized suspensions of human insulin/zinc/protamine complexes (as described for example in U.S. Pat. No. 5,834,422), which make it possible to slow the in vivo release of the protein. In these suspensions, the insulin is complexed with the protamine and the zinc to form a partially crystallized precipitate. After subcutaneous injection, the rate of release of the insulin is controlled by the kinetics of in vivo dissolution of this precipitate and the kinetics of decomplexing of the insulin. The duration of action of this type of insulin, although prolonged compared with that of a rapid insulin, does not exceed 16 hours and therefore does not really cover the nychthemeron. Moreover, a hypoglycemia peak is observed after administration of this long-acting human insulin NPH. The latter is therefore not a true long-acting insulin as defined above.

Recently, patent U.S. Pat. No. 5,656,722 describes a novel protein structure similar to insulin called GLARGINE® contained in a formulation.

A completely different route for obtaining a form for prolonged release of protein is disclosed in patent U.S. Pat. No. 5,904,936 (EP-B-0 734 720).

The technique proposed neither consists in chemically modifying the insulin nor in complexing the insulin with protamine and zinc. It rather involves adsorbing human insulin onto biocompatible nanoparticles formed spontaneously by self-assembly, in water, of amphiphilic polyamino acids such as poly(L-leucine-b-sodium glutamate)—hereinafter called poly(Leu-block-Glu). This self-assembly leads to a colloidal suspension of nanoparticles. Human insulin, while exposed to such a colloidal suspension, is spontaneously adsorbed onto the particles to form a noncovalent insulin/particle complex. After subcutaneous injection, the human insulin becomes gradually dissociated from the complex, making it possible to maintain its plasma concentration at a value close to its basal value for a prolonged period. The advantage of this approach is to use, in a nondenaturing method, unmodified human insulin and a biocompatible polymer, without using potentially denaturing surfactants. It is specified in example 14 of patent U.S. Pat. No. 5,904,936 that human insulin spontaneously combines with nanoparticles of poly(Leu-block-Glu) up to a maximum level of 0.65 mg of human insulin per 10 mg of poly(Leu-block-Glu), that is 6.5% by mass. This injectable long-acting insulin formulation according to example 14 of patent U.S. Pat. No. 5,904,936 can be perfected as regards the following points:
The formulation could be improved in terms of ease of injection, in particular with syringes with fine needles 29 G, 30 G or 31 G, which markedly improve the comfort of the patient knowing that the latter is subjected to a daily injection over several tens of years.
The formulation could be improved in terms of stability in order to further delay the degradation of insulin.
The subcutaneous injection of this formulation in pigs can sometimes lead to the formation of marked edemas and erythemas, resulting in a local tolerance which could be improved, making this formulation hardly compatible with a daily pharmaceutical application over a very long period.
The formulation is difficult to sterilize by filtration.

In its example 9, application WO-A-01/37809 describes an injectable long-acting insulin formulation whose pH is 7.4 and which comprises a suspension of nanoparticles of poly(Leu-block-Glu) polymer. This suspension comprises (per ml of preparation): 80 IU of insulin and 56 mg of polymer, that is an insulin/poly(Leu-block-Glu) ratio by mass of 5%. Administered to beagle dogs in an amount of 2 IU/kg, this suspension results in a prolonged release over 24 hours approximately. However, this formulation has a stability that can be perfected as demonstrated in example 5 below of the present application.

On the strength of this experience, the applicant has redefined specifications for an injectable long-acting human insulin formulation:
1. The formulation could be improved by being more easily injectable through a needle of small diameter (e.g. 29 G, 30 G or 31 G), in order to improve the comfort of the patient and thus compliance with the treatment.
2. The insulin formulation can be perfected in terms of its stability in particular at 4° C. and at room temperature, so as not to cause modifications in the properties of the formulation or degradations of human insulin.
3. The formulation could be greatly improved if it were endowed with excellent local tolerance, so as to be compatible with a daily injection over a period of several tens of years.
4. The bioavailability of the insulin provided by such a formulation could benefit from being as high as possible.
5. The efficacy of the formulation, measured for example by its hypoglycemic effect, could benefit from being as high as possible for at least 24 hours after injection.
6. The formulation could benefit from having a rheology allowing easy filling of the syringe by the patient.
7. The capacity to be sterilized by filtration could be a decisive advantage for the formulation.

Unpublished French patent application No. 04 51578 of Jul. 19, 2004 describes an injectable colloidal suspension of long-acting insulin. This suspension is intended to satisfy the abovementioned specifications. It comprises nanoparticles of at least one poly(Leu-block-Glu), loaded with insulin. Its pH is between 5.8 and 7.0 (e.g. 6.3-6.5). Its osmolarity O (in mOsmol) is such that: 270≦O≦800 (e.g. circa 300). This osmolarity is adjusted by adding at least one salt, for example NaCl or MgCl₂. Its viscosity v (in mPa·s) is such that v≦40 (e.g. ≈25). The nanoparticles of poly(Leu-block-Glu) have a mean hydrodynamic diameter Dh such that: 15≦Dh≦40. The concentration of poly(Leu-block-Glu) is between 30-55 g/l (e.g. 42). The insulin/poly(Leu-block-Glu) ratio is between 5 and 11. The maximum loading rate Ta of the nanoparticles with insulin, expressed in % by mass of insulin combined relative to the mass of poly(Leu-block-Glu) and measured according to a procedure Ma, is such that: 12≦Ta≦25.

This injectable colloidal suspension of long-acting insulin is endowed with very good pharmacokinetic and pharmacodynamic performance features. However, it has emerged that this suspension can be perfected in terms of viscosity. Indeed, for an administration at least once per day for life, it is highly desirable to ensure that this suspension is as fluid as possible, ideally like water. This requirement applies both with respect to the ease of filling of the syringe equipped with a fine needle (pumping/suction) and as regards the ease of injection (expulsion of the suspension).

The expected reduction in viscosity should not be obtained at the expense of the pharmacokinetic and pharmacodynamic performance features of the suspension.

Now, the inventors have demonstrated that the simple solution which would consist in reducing the poly(Leu-block-Glu) concentration in order to reduce the viscosity of the suspension is harmful to the pharmacokinetic and pharmacodynamic performance features.

### BRIEF DISCLOSURE OF THE INVENTION

Faced with this technical problem, the inventors therefore set themselves the main objective of providing a suitable solution thereto.

Another main objective of the invention is to provide long-acting insulin formulations in the form of a colloidal suspension:
which allows easy filling of a syringe through a small diameter needle (for example with the gauge 29 G, 30 G or 31 G)
and/or which can be easily injected through a small diameter needle (for example with the gauge 29 G, 30 G or 31 G), without damaging the therapeutic efficacy of the insulin.

Another main objective of the present invention is to provide a long-acting insulin formulation in the form of a colloidal suspension comprising nanoparticles of poly(Leu-block-Glu) and which is filterable on 0.2 μm filters for sterilization purposes.

Another main objective of the invention is to perfect the suspension disclosed in unpublished French patent application No. 04 51578 of Jul. 19, 2004.

Another main objective of the invention is to fully satisfy the specifications described above.

Another main objective of the invention is to provide a long-acting insulin formulation in the form of a colloidal suspension which maintains a high hypoglycemic effect extending over at least 24 hours after a single administration, for example by the subcutaneous route.

Another main objective is to provide a long-acting insulin formulation in the form of a colloidal suspension which is stable and which does not induce modification of the structure and bioactivity of the insulin.

Another main objective of the invention is to provide long-acting insulin formulations in the form of colloidal suspensions injectable in a small injection volume and high concentration of human insulin, typically 100 IU/ml, and without damaging the therapeutic efficacy and in particular the duration of the hypoglycemic effect.

Another main objective of the present invention is to provide a long-acting insulin formulation in the form of a colloidal suspension having good local tolerance and a safety during use which are compatible with the chronic treatment of diabetics.

Another main objective of the invention is to provide a long-acting insulin formulation in the form of a colloidal suspension in which the insulin is an unmodified human insulin.

Another main objective of the present invention is to provide a long-acting insulin formulation in the form of a colloidal suspension comprising nanoparticles of poly(Leu-block-Glu) onto which the proteins are adsorbed reversibly, noncovalently and without denaturation.

Another main objective of the invention is to provide a method for preparing these colloidal suspensions of long-acting insulin, said method needing to be simple to carry out, nondenaturing for the protein and additionally having to always reliably ensure the reproducibility of the characteristics of the formulation.

The above objectives, among others, are achieved by the present invention which relates, first of all, to an injectable long-acting insulin formulation comprising an aqueous and stable colloidal suspension of nanoparticles based on at least one block polymer of L-leucine and L-sodium glutamate, or poly(L-leucine-b-L-sodium glutamate)—hereinafter called poly(Leu-block-Glu)-, these nanoparticles being loaded with insulin, the pH of this suspension being such that: 6.0≦pH≦7.0;
which comprises at least one magnesium salt in a quantity such that:
   the osmolarity Osm (in mOsmol) is such that: 270≦Osm≦600,
   the viscosity v (in mPa·s), measured according to a procedure Mv, is such that: v≦15,
   the poly(Leu-block-Glu) concentration (in mg/ml) is between 30 and 70, preferably between 38 and 65.

The inventive basis of this novel colloidal suspension capable of constituting an injectable galenic long-acting insulin formulation lies in particular in the combination of the following selections:
osmolarity window ensuring in particular good local tolerance for the formulation;
viscosity window with a ceiling at 15 mPa·s, which participates in imparting on the formulation rheological properties such that it is possible to easily fill a syringe by sucking in the formulation through a small diameter needle (for example with the gauge 29 G, 30 G or 31 G) and to easily inject through these same needles;
nature (Mg²⁺) and quantity of the electrolyte used to adjust the osmolarity.

These advances are obtained without putting a strain on the production cost of this novel formulation.

Advantageously, the poly(Leu-block-Glu) concentration (in mg/ml) of the formulation is between 30 and 70, preferably 38 and 65. This poly(Leu-block-Glu) concentration window makes it possible to preserve the pharmacokinetic and pharmacodynamic performance features of the suspension.

Preferably, the counter-anion for the Mg²⁺ is chosen from the group comprising Cl⁻, SO₄⁻⁻ and mixtures thereof.

Moreover, the formulation according to the invention preferably possesses at least one (ideally all) of the following characteristics:
the particles of the poly(Leu-block-Glu) selected have a mean hydrodynamic diameter Dh, expressed in nanometers (nm) and measured according to a procedure Md, such that:

10≦Dh≦50 preferably, 15≦Dh≦40;
the insulin/poly(Leu-block-Glu) ratio by mass, expressed in %, is such that:

6≦insulin/poly(Leu-block-Glu)≦10,

preferably 3≦insulin/poly(Leu-block-Glu)≦5;
the poly(Leu-block-Glu) concentration (in mg/ml) is between 30 and 70, preferably 38 and 65;
the maximum loading rate Ta of the nanoparticles with insulin, expressed in % by mass of insulin combined relative to the mass of poly(Leu-block-Glu) and measured according to a procedure Ma, is such that:

10≦Ta

preferably, 10≦Ta≦40

and particularly preferably 12≦Ta≦25
the insulin is an unmodified human insulin.

Finally, the injectable long-acting insulin formulation according to the invention is endowed with the following main properties:
a sufficiently low viscosity to have a good capacity for filling a syringe by suction through a small diameter needle and an easy “injectability” through a small diameter needle, which considerably improves the comfort of the patient and therefore increases compliance with the treatment;
this being in combination with good pharmacokinetic and pharmacodynamic performance features, in particular a hypoglycemic activity extending over 24 hours after administering to humans a standard dose of 0.6 IU/kg;
an excellent stability;
a good local tolerance;
a capacity for sterilizing filtration on a 0.2 micron filter by virtue of the small size of the particles.

This suspension additionally has the property of having a low polymer/insulin ratio by mass, leading to a limited additional cost of polymer.

### DETAILED DESCRIPTION OF THE INVENTION

The selection of the parameters defined above in order to obtain a suitable colloidal suspension of insulin as injectable medicinal formulation of long-acting insulin is the fruit of major and long research studies on the measurement and the comparison of the pharmacokinetic and pharmacodynamic activities of these insulin formulations on various animal models and numerous studies on the viscosity (injectable character and capacity to be sucked in through a needle), the stability, the tolerance and the biocompatibility of this particular suspension.

The stable colloidal suspension forming the formulation contains submicron structured nanoparticles formed by self-assembly of a poly(Leu-block-Glu) copolymer.

These nanoparticles are capable:
of spontaneously combining with (adsorbing onto) insulin, noncovalently, in colloidal suspension, in undissolved state and without denaturation, to form a nanoparticle/insulin complex,
and of releasing the insulin, in particular in vivo, in a prolonged and/or delayed manner.

Finally, these nanoparticles are stable in an aqueous phase even in the absence of surfactant(s).

It is to the applicant's credit to have shown that it is possible to reduce the viscosity of the suspension and therefore the force necessary to inject the colloidal suspension through a small diameter needle by varying in particular the choice of the cation which makes it possible to adjust the osmolarity Osm and the concentration C_{Mg2+} of this cation.

It is also to the inventors' credit to have reduced the viscosity by adjusting the osmolarity Osm (in mOsm) of the suspension to a value greater than 270 and less than or equal to 600, by adding at least one salt, and more especially one or more monovalent and/or multivalent (e.g. divalent or trivalent) salts of magnesium.

Thus, C_{Mg2+} between 0.06 and 0.125 mol/l (preferably of the order of 0.08+/−0.01 mol/l) makes it possible to optimize the balance between the duration of action and the injectable character of the suspension.

For the purposes of the present disclosure, the term “of the order” associated with a numerical value means that this value is given with a tolerance which may be up to for example 20%.

This reduced viscosity and therefore this remarkable capacity of the formulation of the invention to be easily injected through a small diameter needle (for example gauge 29 G, 30 G and 31 G or diameter: 0.15 to 0.4 mm, length: 8 to 20 mm) is assessed in particular by means of the force to be exerted on the piston of the syringe. It is for example desirable that this force does not exceed a reasonable value, e.g. of the order of 40 Newtons, preferably of the order of 30 Newtons, and that the flow rate is not less than or equal to 1 ml/min.

The possibility of easily filling the syringe by sucking in the formulation through small diameter needles (for example gauge 29 G, 30 G and 31 G or diameter: 0.15 to 0.4 mm, length: 8 to 20 mm) is also a characteristic sought through the reduction in the viscosity. This operation should for example be typically carried out in a time ≦120 s, preferably ≦60 s and more preferably still ≦30 s, for a volume of 500 μl.

The viscosity is measured according to the procedure Mv described below:

### Procedure Mv

In accordance with the present invention, the determination of the important parameter which the viscosity v (in mPa·s at 20° C.) represents may be carried out, for example, at 20° C. with the aid of a rheometer AR1000 (TA Instruments) equipped with a cone-plate geometry (4 cm, 2°). The viscosity v is measured for a shear gradient of 10 s⁻¹.

The colloidal suspension of nanoparticles according to the invention results from a particular selection among those described broadly in WO-A-01/37809. This particular selection was found after numerous trials aimed at optimizing the contradictory requirements of the specifications mentioned above.

The optimization of the stability of the suspension involves adjusting the pH of the suspension between 5.8 and 7.0, preferably between 6.0 and 7.0, and particularly preferably to a value of the order of 6.5. The stability in question is, on the one hand, a physicochemical stability of the colloidal suspension of nanoparticles of poly(Leu-block-Glu) and, on the other hand, a stability of the insulin active agent in terms of therapeutic efficacy (control of glycemia). Advantageously, the formulation remains stable after 2 years of storage at 5° C., for example.

In accordance with the invention, the nanoparticles have a small size and have more precisely a hydrodynamic diameter Dh, measured according to the procedure Md, such that (in nm) preferably in increasing order: 10≦Dh≦50 and 15≦Dh≦40.

One of the impacts among others of these choices of size of poly(Leu-block-Glu) particles is that the formulation according to the invention can be easily filtered on a sterilizing filter having a pore size of 0.2 μm, which makes it possible to obtain, easily and at a lower cost, a sterile injectable formulation. Moreover, it appears, surprisingly, that the local tolerance of these particles is better than that of larger size, as demonstrated below in the examples.

### Procedure Md:

The poly(Leu-block-Glu) solution, having a concentration of about 75 g/l, is diluted using a 0.15 M aqueous sodium chloride solution so as to obtain in the end a poly(Leu-block-Glu) concentration between 0.5 and 4 g/l, and preferably equal to 2 μl. This suspension is stirred for 1 hour, and then introduced into the diffusion cell of a Brookhaven type light scattering apparatus operating with a laser beam of wavelength 488 nm and vertically polarized. The hydrodynamic diameter is calculated from the self-correlation function of the electric field by the method of cumulus, as described in the manual “Surfactant Science Series” volume 22, Surfactant Solutions, Ed. R. Zana, Chap. 3, M. Dekker, 1984.

According to another preferred characteristic, the poly(Leu-block-Glu)s selected in accordance with the invention have the following characteristic:

the maximum loading rate Ta of the nanoparticles with insulin, expressed in % by mass of insulin combined relative to the poly(Leu-block-Glu) mass and measured according to a procedure Ma, is such that:

10≦Ta

preferably, 10≦Ta≦40

and particularly preferably 12≦Ta≦25.

### Procedure Ma:

(a) Preparation of aqueous solutions of insulin: freeze-dried human recombinant insulin is poured into a volume V of 0.01N hydrochloric acid solution over at most 15 min. This solution is then poured into the same volume V of 0.01N NaOH solution. The pH is adjusted to between 7.2 and 7.4 with a 1N sodium hydroxide solution. The solution is gently stirred for 30 min. The mass of insulin and the volume V are calculated as a function of the desired volume V′ of final solution so as to obtain insulin concentrations of 100 IU/ml, 120 IU/ml and 140 IU/ml.
(b) Preparation of the insulin formulation. The concentrated solution (concentration of about 75 g/l) of poly(Leu-block-Glu) is added to the insulin solutions at the rate of 11 mg/ml. These mixtures are degassed and then placed in a rocking shaker at 25° C. for 2 hours and then degassed again. The pH is adjusted to between 7.2 and 7.4 with a 1N HCl solution and the mixtures are stirred (rocking shaker) overnight at room temperature.
(c) Assay of the free insulin: the formulations are injected onto a size exclusion liquid chromatography column under nondissociating conditions and the free insulin is assayed by fluorimetry.

According to an advantageous variant, the insulin combined with the nanoparticles in the suspension constituting the formulation of the invention is an unmodified insulin.

For the purposes of the invention, an unmodified insulin is a recombinant or nonrecombinant insulin which has not undergone any transformation of its primary structure or any modification of the amino acid side groups.

Advantageously, the suspension constituting the formulation of the invention comprises at least one preservative, preferably selected from the group comprising: phenols, cresols (e.g. meta-cresol), methyl, propyl or butyl para-hydroxybenzoate, or any other preservative known to a person skilled in the art (see for example the article by L. A. Gatlin et al. in Injectable Drug Development, P. K. Gupta eds Interpharm Press, Denver Colo., 1999) and mixtures thereof.

The method for synthesizing the poly(Leu-block-Glu) and the method for producing the nanoparticles of poly(Leu-block-Glu) in aqueous suspension are preferably carried out according to the modalities and the recommendations described in WO-A-01/37809.

Instead of being a stable suspension in an aqueous liquid medium, the nanoparticles of poly(Leu-block-Glu) loaded with insulin could also: exist in a stable solid state, preferably in pulverulent form. Thus, the present invention also relates to a solid—preferably pulverulent—, which comprises poly(Leu-block-Glu)-based nanoparticles loaded with insulin and which is obtained from the liquid suspension which is defined above and which constitutes at least part of the formulation of the invention. This production is carried out by any known and appropriate means such as freeze-drying, spray-drying or drying.

The method for preparing the injectable liquid formulation of long-acting insulin involves poly(Leu-block-Glu) not loaded with insulin and mainly consists
on the one hand,
   i. in mixing, in aqueous liquid medium, at least one poly(Leu-block-Glu) and insulin, preferably with stirring,
   ii. and in optionally adding excipients, if necessary in adjusting the pH to a value of between 6.0 and 7.0;
on the other hand, in adjusting the osmolarity Osm (in mOsmol) of the formulation using at least one magnesium salt, such that:
   270≦Osm≦600;
   the viscosity v (in mPa·s), measured according to a procedure Mv, is such that: v≦15;
   the poly(Leu-block-Glu) concentration (in mg/ml) is between 30 and 70, preferably between 38 and 65;
and optionally in filtering the suspension thus obtained.

Preferably, adjusting the osmolarity Osm (in mOsmol) of the formulation using at least one magnesium salt is carried out such that the C_{Mg2+} concentration (in mol/l) is the following:

0.06≦C_{Mg2+}≦0.125; preferably of the order of 0.08+/−0.01.

Advantageously, the combination of the insulin with the nanoparticles during step (i) is carried out using at least one of the following methods:
a first method mainly consists in exposing an aqueous phase containing the insulin to the colloidal suspension of nanoparticles of poly(Leu-block-Glu);
a second method mainly consists in exposing (by mixing them) the poly(Leu-block-Glu) in the pulverulent state to an aqueous phase containing the insulin, e.g. at a concentration between 100 and 200 IU/ml;
a third method mainly consists in mixing the insulin in the pulverulent state with an aqueous phase containing the poly(Leu-block-Glu).

More precisely, in the first method, a suspension of nanoparticles at neutral or isotonic pH is reconstituted at a concentration of 60 mg/ml or more (according to the concentration desired in the final suspension). A concentrated insulin solution (typically between 500 and 600 IU/ml—pH between 7 and 8—isotonic) is then freshly prepared from insulin powder (dissolution in acidic medium followed by neutralization). The two solutions are mixed by stirring for a few minutes and this phase is optionally followed by a “maturation” phase of a few hours. The pH is then adjusted to a value of between 5.8 and 7.0.

It is important to ensure that at least one of the main characteristics of the invention is preserved, namely the establishment of a pH of between 6.0 and 7.0. This factor greatly contributes to the stability of the suspension of nanoparticles of poly(Leu-block-Glu). The pH may be adjusted by any known and appropriate means, namely in particular by acidification, for example, in the following manner:
-1- addition of 0.1N hydrochloric acid to the suspension loaded with insulin (an intermediate precipitation then occurs which disappears after stirring for about one hour)
-2- addition of 0.1N acetic acid to the suspension loaded with insulin (no intermediate precipitation).

The addition -2- of acetic acid is preferred.

During the preparation of the formulation according to the invention, excipients may be optionally added, and if necessary the pH may be readjusted to a value of between 5.8 and 7.0, and the suspension thus obtained may be optionally sterilized by filtration on pores of 0.2 microns.

These other excipients may be in particular at least one preservative, preferably selected from the group comprising: phenols, cresols (e.g. meta-cresol), methyl, propyl or butyl para-hydroxybenzoates, or any other preservative known to persons skilled in the art (see for example the article by L. A. Gatlin et al. in Injectable Drug Development, P. K. Gupta eds Interpharm Press, Denver Colo., 1999) and mixtures thereof.

The mixing conditions, both for regulating the pH and for adding the excipients, are standard and within the capability of persons skilled in the art, in particular in terms of temperature, pressure and stirring.

Finally, the liquid suspension may be converted to a pulverulent solid by any conventional method known to persons skilled in the art, such as for example freeze-drying, spray-drying or drying.

More generally, the invention also relates to a method for preparing an injectable formulation of protein(s), which mainly consists
on the one hand, in mixing at least one protein with an aqueous liquid medium, in optionally adding excipients, if necessary in adjusting the pH to a value of between 6.0 and 7.0;
and on the other hand, in adjusting the osmolarity of the formulation using at least one magnesium salt, such that:

270≦Osm≦600;
the viscosity v (in mPa·s), measured according to a procedure Mv, is such that:

v≦15;
the poly(Leu-block-Glu) concentration (in mg/ml) is between 30 and 70, preferably between 38 and 65;
and optionally in filtering the mixture thus obtained.

The expression “protein” denotes any polyamino acid, for example oligopeptide, polypeptide [e.g. poly(Leu-block-Glu)], protein strictly speaking [e.g. insulin].

According to another of its aspects, the invention relates to the pharmaceutical and veterinary applications of the poly(Leu-block-Glu)-insulin suspension. The main application is the treatment of diabetes, and more precisely of type I and type II diabetes.

Thus, the subject of the invention is also a medicament which comprises formulation comprising a poly(Leu-block-Glu)-insulin colloidal suspension, this formulation being as defined above and/or formulation obtained by the method which is itself also described above and/or pulverulent solid as defined above.

It is preferably a medicament intended for the treatment of diabetes and more precisely of type I and type II diabetes.

According to an advantageous feature of the invention, the medicament to which it relates is similar to an injectable liquid formulation of long-acting human insulin capable of providing the diabetic patient, after a subcutaneous injection, with a basal insulin concentration for at least 24 hours, in a repeated injection regimen.

The basal insulin concentration should be understood to mean, in the present disclosure, a typical concentration in the blood observed in a healthy individual, that is 30 picomol/l.

The invention additionally relates to a method for treating diabetes, in, particular type I and II diabetes, which mainly consists in administering to the patient the abovementioned medicament based on a formulation comprising a stable aqueous colloidal suspension of poly(Leu-block-Glu) nanoparticles loaded with insulin, preferably unmodified.

It is preferably a daily administration by injection, preferably subcutaneous injection.

The medicament according to the invention may also be provided in the form of a pulverulent solid described above, and optionally of the aqueous liquid for the preparation of the suspension.

The result is that the invention also covers a galenic presentation comprising, on the one hand, pulverulent solid as defined above and, on the other hand, separately, aqueous liquid for the reconstitution, before administration, of the suspension constituting the formulation according to the invention.

The examples which follow will make it possible to better understand the invention in its various product/method/application aspects. These examples illustrate the preparation of the formulation according to the invention based on a suspension of poly(Leu-block-Glu) nanoparticles loaded with insulin, and they present the structural characteristics and the properties of this formulation by comparing it to the poly(Leu-block-Glu)-insulin formulations according to the prior art.

### EXAMPLES

### Example 1

### Preparation of a Long-Acting Insulin Suspension

1.1—Preparation of a Concentrated Intermediate Colloidal Suspension of poly(L-leucine-block-L-sodium glutamate) (P) Nanoparticles at 60 mg/g:

The preparation of the suspension is carried out under a laminar flow cabinet or in a sterile room. 159 g of poly(Leu-block-Glu) polymer in solution in water at 73.9 mg/g are successively introduced into a glass bottle. 1.9 g of 30% NaCl and 0.1 g of 1N NaOH are then successively added to the solution in order to adjust the pH to 7.4 and the osmolarity to 300 mOsm/kg and to bring the polymer concentration to 60.5 mg/ml.

### 1.2—Preparation of an Intermediate Insulin Solution at 590 IU/ml:

7 g of recombinant human insulin (powder) with an activity of 27.5 IU/g and containing 3.8% moisture are introduced into a glass bottle, 149.5 g of water for injection are added and the insulin is dispersed with slow magnetic stirring. 51.20 g of 0.1N HCl are added until a clear acidic insulin solution is obtained. 7.7 g of 0.1N sodium hydroxide are then added so as to obtain a final solution having a pH of between 7.2 and 7.6. 9.4 g of 30% NaCl are added in order to adjust the osmolarity. The solution is then diluted by adding 0.9% (15.1 g) NaCl in order to obtain a final insulin concentration of 590 IU/ml.

The solution is filtered on a 0.2 μm polyethersulfone membrane before mixing with the suspension of nanoparticles.

### 1.3—Preparation of a Solution of Excipients (140 Mm Phenol; 140 mM M-Cresol, 0.1N Acetic Acid):

The following are successively added to a 1 l bottle:
13.2 g of phenol (M=94 g/mol)
100 g of water
15.1 g of m-cresol (M=108 g/mol)
100 g of 1N acetic acid
771.7 g of water.

The solution is stirred for at least half an hour until a clear solution is obtained and then filtered on 0.2 μm PVDF (polyfluorovinylidene) or PTFE (polytetrafluoroethylene) membrane.

### 1.4—Preparation of a Negative Control Comparative Formulation (Formulation CompA) Comprising the Colloidal Suspension of Long-Acting Insulin P of 1.1 Above:

(Osmolarity Osm=300 mOsm; pH=6.5; poly(Leu-block-Glu) concentration Cpol=42 mg/ml; insulin concentration C insulin=3.5 mg/ml)

800 g of suspension P are introduced into a 1 liter bottle.

196 g of solution containing 590 IU/ml of insulin are added under slow magnetic stirring. The stirring is maintained for 15 hours at 25° C. 171 g of solution of excipients are then added to the mixture and the solution is kept stirring for 1 hour. The formulation thus obtained is filtered on a 0.2 μm polyethersulfone membrane.

### Example 2

### Preparation of a Negative Control Comparative Formulation (Formulation CompB) Comprising a Colloidal Suspension of Long-Acting Insulin P of example 1.1

(Osmolarity Osm=450 mOsm; pH=6.5; poly(Leu-block-Glu) concentration Cpol=42 mg/ml; insulin concentration C insulin=3.5 mg/ml)

Formulation B is prepared in a manner similar to example 1, the polymer concentration being adjusted to 60 mg/ml and the osmolality Osm to 507 mOsm/kg in step 1.1.

### Example 3

### Preparation of a Formulation According to the Invention (Formulation C) Comprising a Colloidal Suspension of Long-Acting Insulin P of Example 1.1

(Osmolarity Osm=450 mOsm; pH=6.5; poly(Leu-block-Glu) concentration Cpol=42 mg/ml; insulin concentration C insulin=3.5 mg/ml)

Formulation C is prepared in a manner similar to example 1, the osmolality Osm being adjusted to 507 mOsm/kg in step 1.1 by adding an MgCl₂ solution at 32%.

### Example 4

### Preparation of a Formulation According to the Invention (Formulation CompD) Comprising a Colloidal Suspension without Insulin

(Osmolarity Osm=450 mOsm; pH=7.4; poly(Leu-block-Glu) concentration Cpol=45 mg/ml; insulin concentration C insulin=0 mg/ml)

The preparation of the suspension is carried out under a laminar flow cabinet or in a sterile room. 65.6 g of poly(Leu-block-Glu) polymer in solution in water at 73.5 mg/g are successively introduced into a glass bottle. 2.21 g of 30% NaCl and 0.53 g of 1N NaOH are then successively added to the solution in order to adjust the pH to 7.4 and the osmolarity to 450 mOsm/kg. 2.3 g of 30% NaCl and 37.88 g of water for injection are then added in order to adjust the polymer concentration to 45 mg/ml while maintaining the osmolality at 450 mOsm/kg.

### Example 5

### Preparation of a Formulation According to the Invention (Formulation E) Comprising a Colloidal Suspension without Insulin

(Osmolarity Osm=450 mOsm; pH=7.4; poly(Leu-block-Glu) concentration Cpol=45 mg/ml; insulin concentration C insulin=0 mg/ml)

The preparation of the suspension is carried out under a laminar flow cabinet or in a sterile room. 64.5 g of poly(Leu-block-Glu) polymer in solution in water at 73.5 mg/g are successively introduced into a glass bottle. 2.1 g of 32% MgCl₂ and 0.63 g of 1N NaOH are then successively added to the solution in order to adjust the pH to 7.4 and the osmolarity to 450 mOsm/kg. 1.35 g of MgCl₂, 0.96 g of 30% NaCl and 37.32 g of water for injection are then added in order to adjust the polymer concentration to 45 mg/ml while maintaining the osmolality at 450 mOsm/kg.

### Example 6

### Preparation of a Formulation According to the Invention (Formulation F) Comprising a Colloidal Suspension of Long-Acting Insulin P of Example 1.1

(Osmolarity Osm=450 mOsm; pH=6.5; poly(Leu-block-Glu) concentration Cpol=45 mg/ml; insulin concentration C insulin=3.5 mg/ml)

Formulation F is prepared in a manner similar to example 1, the polymer concentration being adjusted to 64.7 mg/ml and the osmolality Osm to 535 mOsm/kg in step 1.1.

### Example 7

### Characteristics of the Formulations CompA, CompB, C, CompD, E, F of Long-Acting Insulin According to the Invention (cf. Table 1)

The viscosity measurements are carried out at 20° C. on a rheometer AR1000 (TA Instruments) equipped with a cone-plate geometry (4 cm, 2°). The viscosity is measured for a shear gradient of 10 s⁻¹.

TABLE 1

| | [Polymer] | Osmolality | Viscosity | [NaCl] | [MgCl₂] | [Insulin] |
|---|---|---|---|---|---|---|
| | mg/ml | mOsm/kg | mPa · s | mol/l | mol/l | IU/ml |
| Formulation | 42 | 306 | 15 | 0.085 | 0 | 97 |
| compA | | | | | | |
| Formulation | 42 | 450 | 10 | 0.157 | 0 | 102.7 |
| compB | | | | | | |
| Formulation | 42 | 472 | 4 | 0.024 | 0.085 | 100 |
| C | | | | | | |
| Formulation | 44.4 | 448 | 15 | 0.168 | 0 | 0 |
| compD | | | | | | |
| Formulation | 45.5 | 452 | 5 | 0.023 | 0.09 | 0 |
| E | | | | | | |
| Formulation | 44.2 | 462 | 5 | 0.027 | 0.08 | 104.3 |
| F | | | | | | |

The magnesium chloride used in accordance with the invention (formulations C, E and F) is particularly advantageous because, for a similar osmolality, it allows greater reduction in viscosity: 4 and 5 mPa·s for formulations C, E and F respectively against 10 and 15 mPa·s for formulations compB and compD respectively.

### Example 8

### Filling Time for Formulations CompA, CompB and C

The time for filling a syringe with insulin through a 30 gauge and 8 mm long needle is measured for the various formulations described in the preceding examples.

The filling volume is 0.5 ml corresponding to 50 IU/ml.

The following results, presented in table 2 below, are obtained:

TABLE 2

| Formulation No. | Filling time (s) |
|---|---|
| compA | 50 |
| compB | 35 |
| C | 15 |

The use of a hyperosmotic solution adjusted in terms of magnesium chloride (formulation C) makes it possible to reduce the time for filling a syringe to a sufficiently low period to allow easy daily use by the patients.

### Example 9

### Injection Time for Formulations CompA, CompB and C

The insulin-based products are subcutaneously injected either by means of insulin needles, or by means of an insulin pen. The time for injecting a 50 IU dose for various formulations is measured for a constant force of 20 Newtons. The pen used is a NOVOPEN III, using 3 ml cartridges and 31 G “thin wall” needles of the Becton Dickinson brand.

TABLE 3

| Formulation No. | Time for injecting a 50 IU dose (s) |
|---|---|
| compA | >60 |
| compB | 20 |
| C | 12 |

The use of a hyperosmotic solution adjusted in terms of magnesium chloride (formulation C) makes it possible to bring the injection time for a syringe to a time which is sufficiently short to easily allow daily use by the patients.

### Example 10

### Local Tolerance—Formulations CompD, E and F

The model used is a pig skin in vivo model whose high sensitivity compared with a human skin has been shown for the type of product studied.

The local tolerances for formulations compD, E and F were compared in this model.

Formulation compD comprises:
45 mg/ml of poly(Leu-block-Glu) polymer
an osmolality of 450 mOsm adjusted with sodium chloride and is prepared according to the procedure described in example 4 above.

Formulation E comprises:
45 mg/ml of poly(Leu-block-Glu) polymer
an osmolality of 450 mOsm adjusted with magnesium chloride and is prepared according to the procedure described in example 5 above.
   Formulation F according to the invention comprises:
100 IU/ml of human recombinant insulin
45 mg/ml of poly(Leu-block-Glu) polymer
21 mM of phenol and meta-cresol
an osmolality of 450 mOsm adjusted with magnesium chloride and is prepared according to the procedure described in example 6 above.

An NaCl/MgCl₂ 450 mOsm formulation is used as negative reference in the study.

The various products were subcutaneously injected under calibrated conditions, in a volume of 0.50 ml, under the skin of the belly of 9 domestic pigs (Seghers hybrid×Landrace weighing 50 to 60 kg). The clinical signs—erythema and edema and induration—were then evaluated for the three days which followed the injection.

The negative reference (NaCl/MgCl₂ 450 mOsm) did not cause a clinical reaction. Formulation compD caused a weak and transient local reaction. The reaction disappears within about 3 days.

Formulations E and F caused a local reaction of the same type and of the same intensity as that observed for formulation compD.

Thus, in this very sensitive model, formulation E adjusted in terms of magnesium chloride exhibits a local tolerance equivalent to that of formulation compD adjusted in terms of sodium chloride. This good local tolerance is confirmed with formulation F comprising magnesium chloride and insulin. This local tolerance is perfectly suited to daily administration of the medicament.

### Example 11

### In Vivo Trial—Formulations CompA and C

A study was carried out so as to compare the pharmacodynamics (i.e. the glycemia) of formulation CompA, corresponding to example 1 above, and of a poly(Leu-block-Glu) formulation (formulation C according to the invention) containing magnesium chloride described in example 3.

Formulation CompA comprises:
100 IU/ml of human recombinant insulin
42 mg/ml of poly(Leu-block-Glu) polymer
21 mM of phenol and meta-cresol
   and is prepared according to example 1 above.

Formulation C according to the invention comprises:
100 IU/ml of human recombinant insulin
42 mg/ml of poly(Leu-block-Glu) polymer
21 mM of phenol and meta-cresol
   and is prepared according to example 3 above.

Formulations CompA and C were subcutaneously administered at the dose of 1 IU/kg to 12 and 16 beagle dogs, respectively, that had been starved for 16 hours. The plasma glucose concentrations were then evaluated (Advia 1650 automated machine, Bayer Diagnostics) over a period of 32 hours after administration in order to determine the pharmacodynamic parameters Cmin, AUC₀₋₃₂ₕ and T50%_{AUC}, presented in table 4. The standard deviations are indicated in brackets in table 4.
SSC₀₋₃₂ₕ, expressed as percentages of the basal glucose level in the plasma, represents the area between the plasma glucose profile as a function of time and the basal plasma glucose level, from 0 to 32 hours. This parameter is calculated by the so-called trapezoidal method.
Cmin, expressed as percentages of the basal glucose level in the plasma, represents the minimum glucose concentration observed in the plasma.
T50% SSC, expressed in hours, represents the time necessary to obtain 50% of the SSC₀₋₃₂ₕ.

TABLE 4

| | | | T50%_{AUC} |
|---|---|---|---|
| Formulation No. | Cmin (%) | AUC_{0-32 h} (%*h) | (h) |
| A | 38 (8) | 827 (205) | 9 (2) |
| C | 40 (9) | 769 (166) | 9 (2) |

It is observed that formulations compA and C according to the invention make it possible to obtain similar pharmacodynamic parameters. Formulation C according to the invention therefore has an identical efficacy to that of formulation compA, while having significantly improved filling and injection times compared with compA.
21 mM of phenol and meta-cresol
   and is prepared according to example 1 above.

Formulation C according to the invention comprises:
100 IU/ml of human recombinant insulin
42 mg/ml of poly(Leu-block-Glu) polymer
21 mM of phenol and meta-cresol
   and is prepared according to example 3 above.

Formulations CompA and C were subcutaneously administered at the dose of 1 IU/kg to 12 and 16 beagle dogs, respectively, that had been starved for 16 hours. The plasma glucose concentrations were then evaluated (Advia 1650 automated machine, Bayer Diagnostics) over a period of 32 hours after administration in order to determine the pharmacodynamic parameters Cmin, AUC₀₋₃₂ₕ and T50%_{AUC}, presented in table 4. The standard deviations are indicated in brackets in table 4.
SSC₀₋₃₂ₕ, expressed as percentages of the basal glucose level in the plasma, represents the area between the plasma glucose profile as a function of time and the basal plasma glucose level, from 0 to 32 hours. This parameter is calculated by the so-called trapezoidal method.
Cmin, expressed as percentages of the basal glucose level in the plasma, represents the minimum glucose concentration observed in the plasma.
50% SSC, expressed in hours, represents the time necessary to obtain 50% of the SSC₀₋₃₂ₕ.

TABLE 4

| | | | T50%_{AUC} |
|---|---|---|---|
| Formulation No. | Cmin (%) | AUC_{0-32 h} (%*h) | (h) |
| A | 38 (8) | 827 (205) | 9 (2) |
| C | 40 (9) | 769 (166) | 9 (2) |

It is observed that formulations compA and C according to the invention make it possible to obtain similar pharmacodynamic parameters. Formulation C according to the invention therefore has an identical efficacy to that of formulation compA, while having significantly improved filling and injection times compared with compA.

1. An injectable long-acting insulin formulation comprising an aqueous and stable colloidal suspension of nanoparticles based on at least one poly(Leu-block-Glu) and loaded with insulin, the pH of this suspension being such that: 6.0≦pH≦7.0;
which comprises at least one magnesium salt in a quantity such that:
the osmolarity Osm (in mOsmol) is such that: 270≦Osm≦600,
the viscosity v (in mPa·s), measured according to a procedure Mv, is such that: v≦15.
the poly(Leu-block-Glu) concentration (in mg/ml) is between 30 and 70, preferably between 38 and 65.

2. The formulation as claimed in claim 1 or 2, wherein the counter-anion of Mg²⁺ is chosen in the group comprising Cl⁻, SO4⁻⁻ and mixtures thereof.

3. The formulation as claimed in claim 1, wherein the particles of the poly(Leu-block-Glu) selected have a mean hydrodynamic diameter Dh, expressed in nanometers (nm) and measured according to procedure Md, such that:
10≦Dh≦50
preferably, 15≦Dh≦40.

4. The formulation as claimed in any one of the preceding claims, wherein the insulin/poly(Leu-block-Glu) ratio by mass, expressed in %, is such that:
6≦insulin/poly(Leu-block-Glu)≦10.

5. The formulation as claimed in any one of the preceding claims, wherein the maximum loading rate Ta of the nanoparticles with insulin, expressed in % by mass of insulin combined relative to the mass of poly(Leu-block-Glu) and measured according to a procedure Ma, is such that:
10≦Ta
preferably, 10≦Ta≦40
and particularly preferably 12≦Ta≦25.

6. The formulation as claimed in any one of the preceding claims, wherein the insulin is an unmodified human insulin.

7. The formulation as claimed in any one of the preceding claims, which comprises at least one preservative, preferably selected from the group comprising: phenols, cresols, methyl, propyl or butyl para-hydroxybenzoates and mixtures thereof.

8. A pulverulent solid, which comprises nanoparticles of poly(Leu-block-Glu) loaded with insulin and which is obtained from the formulation as claimed in any one of claims 1 to 7.

9. A method for preparing an injectable long-acting insulin formulation, in particular as claimed in any one of claims 1 to 7, this formulation comprising an aqueous and stable colloidal suspension of nanoparticles of at least one poly(L-leucine-b-L sodium glutamate) hereinafter called poly(Leu-block-Glu), loaded with insulin,
which mainly consists
on the one hand,
i. in mixing, in aqueous liquid medium, at least one poly(Leu-block-Glu) and insulin, preferably with stirring,
ii. and in optionally adding excipients, if necessary in adjusting the pH to a value of between 6.0 and 7.0;
on the other hand, in adjusting the osmolarity Osm (in mOsmol) of the formulation using at least one magnesium salt, such that:
270≦Osm≦600;
the viscosity v (in mPa·s), measured according to a procedure Mv, is such that: v≦15;
the poly(Leu-block-Glu) concentration (in mg/ml) is between 30 and 70, preferably between 38 and 65;
and optionally in filtering the suspension thus obtained.

10. The method as claimed in claim 9, wherein adjusting, using at least one magnesium salt, the osmolarity Osm (in mOsmol) of the formulation is carried out such that the CMg2+ concentration (in mol/l) is the following:
0.06≦C_{Mg2+}≦0.125; preferably of the order of 0.08+/−0.01.

11. A method for preparing an injectable formulation of protein(s), which mainly consists:
on the one hand, in mixing at least one protein with an aqueous liquid medium, in optionally adding excipients, if necessary in adjusting the pH to a value of between 6.0 and 7.0;
and on the other hand, in adjusting the osmolarity of the formulation using at least one magnesium salt, such that:
270≦Osm≦600;
the viscosity v (in mPa·s), measured according to a procedure Mv, is such that: v≦15;
the poly(Leu-block-Glu) concentration (in mg/ml) is between 30 and 70, preferably between 38 and 65;
and optionally in filtering the mixture thus obtained.

12. The method as claimed in claim 9, wherein the combination of the insulin with the nanoparticles during step (i) is carried out using at least one of the following methods:
a first method mainly consists in exposing an aqueous phase containing the insulin to the colloidal suspension of nanoparticles of poly(Leu-block-Glu);
a second method mainly consists in exposing (by mixing them) the poly(Leu-block-Glu) in the pulverulent state to an aqueous phase containing the insulin;
a third method mainly consists in mixing the insulin in the pulverulent state with an aqueous phase containing the poly(Leu-block-Glu).

13. A medicament, which comprises injectable long-acting insulin formulation as claimed in any one of claims 1 to 7 and/or formulation obtained by the method as claimed in any one of claims 9 to 12 and/or pulverulent solid as claimed in claim 8.

14. The medicament as claimed in claim 13, which is intended for the treatment of diabetes.

15. The medicament as claimed in claim 13 or 14, which is susceptible of providing the diabetic patient, after a subcutaneous injection, with a basal insulin concentration for at least 24 hours.

16. The medicament as claimed in any one of claims 13 to 15, which consists of a galenic presentation comprising, on the one hand, pulverulent solid as claimed in claim 8 and, on the other hand, separately, aqueous liquid for the reconstitution, before administration, of the formulation as claimed in any one of claims 1 to 7 and/or of the formulation obtained by the method as claimed in any one of claims 9 to 12.
